(19) United States
(12) Patent Application Publication
(10) Pub. No.: US 2019/0287039 A1
(43) Pub. Date: Sep. 19, 2019

(54) SYSTEM AND METHOD FOR DYNAMICALLY EVALUATING SERVICE PROVIDER PERFORMANCE

(71) Applicant: Consolidated Research, Inc., Los Angeles, CA (US)

(72) Inventor: Gregory Ridgeway, Washington, DC (US)

(21) Appl. No.: 16/343,740

(22) PCT Filed: Oct. 20, 2017
(86) PCT No.: PCT/US2017/057694

§ 371 Date: Apr. 19, 2019

Related U.S. Application Data
(60) Provisional application No. 62/410,817, filed on Oct. 20, 2016.

(51) Int. Cl. G06Q 10/06; G16H 40/20; G06F 16/2455; G06F 17/18

(52) U.S. Cl. CPC G06Q 10/06393; G06F 17/18; G06F 16/2455; G16H 40/20

(57) ABSTRACT

Systems and methods for dynamically evaluating service provider performance are provided, including constructing benchmarks for analyzing service providers against one another, which account for the characteristics of the given service provider's cases, services, patients, or clients to ensure that the benchmark contains cases, services, patients, or clients with a similar set of characteristics. Underperforming and/or overperforming service providers may be compared relative to each other, based on their performance relative to their individual benchmarks. The systems and methods provide reporting on the results for each service provider, including a report card listing observed outcomes, its benchmark outcomes, and an outlier probability. Dynamic updating of the service provider benchmarks, outlier probability, and report card occurs, as additional records appear for each service provider, wherein benchmarks may be

### CROSS-REFERENCE TO RELATED APPLICATIONS

This application claims priority to and the benefit of U.S. Provisional Patent Application Ser. No. 62/410,817, filed Oct. 20, 2016 and entitled “SYSTEM AND METHOD FOR BENCHMARKING SERVICE PROVIDERS,” the entire content of which is incorporated herein by reference.

### FIELD OF THE INVENTION

Aspects of example embodiments of the present invention relate generally to methods and systems for the statistical analysis of retrospective service provider data for evaluating the effects of the performance of specific providers among a collection of service providers, and more specifically to the analysis of medical data for evaluating the performance of physicians, clinics, hospitals, and other medical providers.

### BACKGROUND

In many service provider industries, it is often desirable to have available a comparison between different providers across a range of different performance metrics to evaluate the performance of specific service providers. Such industries can include medical (e.g., physicians, clinics, hospitals and the like), emergency responders (e.g., fire and police), education (e.g. teachers and principals), and telecommunications (e.g., wireless phone service providers, internet service providers), as well as many others.

Comparisons between different service providers, however, have been limited due to differences among the basic services rendered by each provider, and/or among the quality or frequency of the services provided. Without a controlled mechanism for benchmarking service providers, it is difficult to determine if, for example, a low value in the performance metric of a given provider is due to a lack of skill or errors committed by that service provider, or if the low performance is due to a relatively high number of occurrences, or due to difficult or extreme circumstances, as compared to other service providers.

As one example, presently, medical providers are evaluated based on basic summary measures such as frequency of prescribing opiates, frequency of patients being readmitted to a hospital within 30 days of release, frequency of prescribing expensive durable medical equipment, frequency of infections, and other quality and performance measures. The comparison of medical providers typically does not account for basic patient differences, and when it does, such comparisons are limited to adjusting merely for age and gender of patients. In the case of the frequency of prescribing opiates, a medical provider that handles a large caseload of patients with pain management issues will naturally prescribe more opiate medications than a medical provider with fewer such patients. Therefore, a comparison between medical providers depends on the ability to compare a given provider's patients' outcomes with the outcomes of a similar collection of patients treated by other medical providers.

### SUMMARY OF THE INVENTION

The present disclosure provides systems and methods for constructing an appropriate benchmark of a given service provider in order to compare the outcomes or results of that provider against the outcomes of a similar collection of results from other service providers. The systems and methods account for the characteristics of the given service provider's cases, services, patients, and/or clients to ensure that the benchmark contains cases, services, patients, and/or clients with a similar set of characteristics. Underperforming and/or overperforming service providers may be compared relative to each other, based on their performance relative to their individual benchmarks. In some embodiments, there is provided a software component system and method for the statistical analysis of patient medical records for the purpose of dynamically benchmarking the performance of medical providers. The system provides a high quality benchmark that matches patient data of a given medical provider with a collection of patient data from other medical providers involving similar diagnoses, medical records, and prescription drug histories. The benchmark includes creating a propensity scoring model, which weights the data for each patient treated by other service providers to collectively resemble the hospital for which the benchmark is being constructed, a regression model providing an estimate of the effects of the service provider on outcomes, a doubly robust estimate that measures the effect of the service provider on the identified effect.

In some embodiments, the system and methods provide mechanisms for dynamically updating the benchmark as new patient records join the data systems, and as providers offer new treatments. To save on the computational cost of continually updating the benchmark for each service provider for each record that is added, the benchmark may be recomputed only when needed. Service providers may be queued for updating their benchmarks, or for analyzing whether an update is necessary, based on a refresh priority score that measures how important is to check whether a given provider's benchmark should be updated. In some embodiments, the quality of the benchmark may be measured to determine if the benchmark is within a specified or user-defined threshold or tolerance. If the quality of the benchmark is within the threshold and therefore the benchmark is sufficient, the original benchmark is used and the propensity score model is not updated. If the quality of the benchmark has deteriorated beyond the threshold, the benchmark is insufficient and the propensity score model is recomputed.

In some embodiments, for each service provider, a report card is created listing the service provider's observed patient outcomes, benchmark outcomes, and outlier probability. For each outcome, a report card listing providers with high outlier probabilities can be created.

### BRIEF DESCRIPTION OF THE FIGURES

FIG. 1 illustrates a schematic block diagram of a system for comparing service providers based on dynamically updated observational data according to one embodiment;

FIG. 2 is a flow diagram of steps performed using a service provider benchmark system according to one embodiment;

FIG. 3 is a flow diagram of steps for a data analysis initialization of the benchmark system of FIG. 1;

FIG. 4 is a flow diagram of steps for dynamic updating of a service provider benchmark system according to one embodiment;

FIG. 5 is a table showing a summary of a sample used for benchmarking hospitals;

FIGS. 6-8 are tables showing samples of patient features comparing one hospital from FIG. 5 to its benchmark;

FIG. 9 depicts a distribution of age for one hospital from FIG. 5 compared to its benchmark;

FIG. 10 depicts a three-way interaction effect for one hospital from FIG. 5 compared to its benchmark;

FIG. 11 depicts a comparison of the mortality rate within 30 days of discharge for the hospitals in FIG. 5 compared to their benchmarks;

FIG. 12 depicts a comparison of the readmission rate within 30 days of discharge for the hospitals in FIG. 5 compared to their benchmarks; and

FIGS. 13-14 depict comparisons of benchmarking and false discovery rates for the hospitals in FIG. 5 as compared with traditional regression models.

### DETAILED DESCRIPTION

Hereinafter, example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof may not be repeated.

The systems and methods described herein provide a benchmark for analyzing service providers against one another. For each service provider, a benchmark is created whereby a first service provider's cases, services, patients, and/or clients can be compared to a dataset containing a collection of cases, services, patients, and/or clients having similar characteristics as the first service provider, but were treated by other service providers. Accounting for the characteristics of the first service provider's cases, services, patients, or clients assures that the benchmark contains cases, services, patients, or clients with a similar set of characteristics. The process can be repeated for each service provider such that multiple benchmarks are established, one per service provider. Each benchmark will have characteristics that are targeted to the service provider under test for that benchmark. For each service provider, a comparison may be created for various observed outcomes for the service provider's cases, services, patients, or clients relative to the benchmark for that service provider. The benchmark comparison may be used to simultaneously compare many different service providers, while adjusting for differences between the cases, services, patients, or clients seen by the various providers. Underperforming and/or overperforming service providers may be compared relative to each other, based on their performance relative to their individual benchmarks. This enables a determination for whether observed differences in outcomes between service providers is due to systematic differences in the service providers themselves, or whether observed differences in outcomes is due to a service provider having a different mix of cases than other service providers. In addition, the systems and methods can provide reporting on the results for each service provider, including a report card listing observed outcomes, its benchmark outcomes, and an outlier probability. Additionally, the system and methods provide for dynamic updating of the service provider benchmarks, outlier probability, and report card, as additional records appear for each service provider. The process for updating the benchmarks can be performed only when needed to save on the computational expense of the benchmarking process. Prior to updating a benchmark for a given service provider, the quality of the benchmark relative to the characteristics of the service provider may be analyzed (including both old records already accounted for in the benchmark and new records since the benchmark was created), to determine whether the benchmark quality has deteriorated beyond a set (e.g., user defined) threshold. If so, a new benchmark may be created for the service provider.

The process of benchmarking allows for more detailed or accurate comparisons of service providers than comparisons that use only averages (such as national averages) to compare service providers to each other without accounting for the unique mix of cases, services, patients, or clients of the particular service providers. For example, using only a national average to compare service providers may show outlier providers in outcomes relative to the national average, but may not account for whether the differences in outcomes is due to a systematic difference in the service providers, or is a product of a given service provider having more outlier cases than average, or a combination of the two.

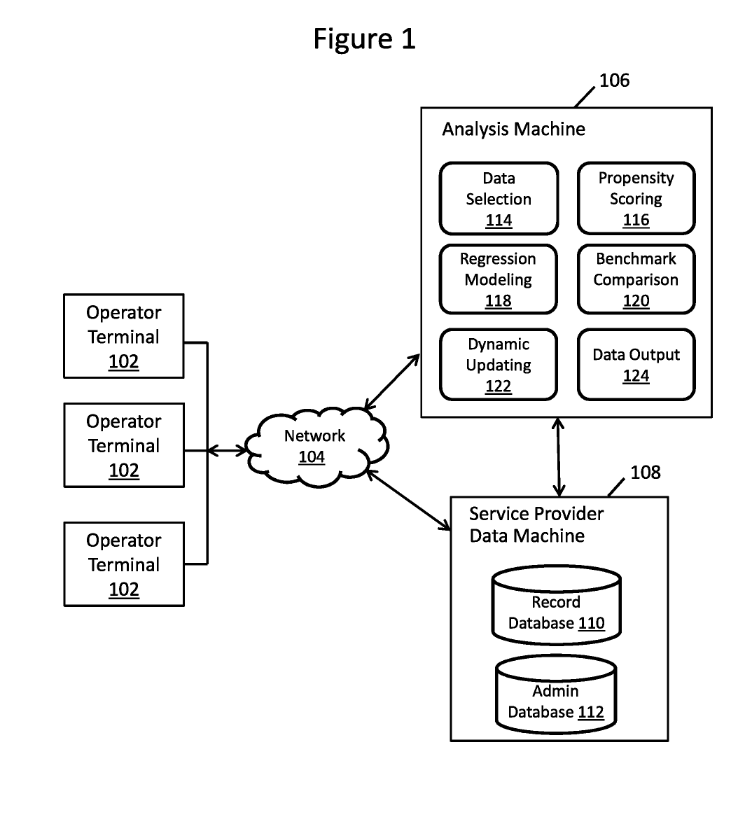

The systems and methods herein include a benchmark comparison using a propensity scoring, a weighted regression model, and an outlier probability. FIG. 1 illustrates a schematic block diagram of a system for comparing service providers based on dynamically updated observational data according to one embodiment. As shown in FIG. 1, the system includes one or more operator terminals 102 for providing system access to operators over a data communications network 104 to an analysis machine 106 and a service provider data machine 108. The service provider data machine 108 may provide access for an operator to a service provider record database 110 and an admin database 112. The analysis machine 106 may include a processor, a non-volatile memory device operably coupled to the processor storing programming instructions and other data, the processor operable to execute program instructions, and a network connection to permit the analysis machine 106 to receive input from the service provider data machine 108 and other sources and to output results to the operator terminal 102 and other destinations.

The service provider record database 110 stores observational service provider data. In one embodiment, the service provider record database 110 includes data from a plurality of different medical providers (such as hospitals), including data from patient medical records for each hospital such as data on categorical features (such as percentage of male or female patients, age distributions of patients, race, occupation, city of residence, prior diagnoses, prior prescriptions, reasons for admission, and the like), numerical measurements (such as temperature, blood pressure, and cholesterol levels, and the like), and patient outcomes (such as hospital readmission within 30 days). The service provider record database 110 may be continually or intermittently updated with additional service provider data over time, for example, as existing hospitals in the database treat additional patients, or as the hospitals have updated records for existing patients, or as new hospitals are to be included in the database.

The admin database 112 stores information related to the operation of the system such as information about what records have been retrieved from the patient record database and when they were retrieved, system data formatting rules, and other data pertinent to the analysis of the service provider record data.

The analysis machine 106 may provide access for an operator to several components including a data selection component 114, a propensity scoring component 116, a regression modeling component 118, a benchmark comparison component 120, a dynamic updating component 122, and a data output component 124. These components may take the form of computer instructions stored in computer memory and executed by a computer processor.

The data selection component 114 presents the operator with an interface for the selection of relevant service provider data and attributes from the service provider record database 110, and retrieves and formats the selected data for use in the propensity scoring component 116.

The propensity scoring component 116 determines and assigns a propensity score to each service provider. In the example of benchmarking for medical services, the propensity score represents the likelihood of a patient in the database being treated by a given medical provider being benchmarked. The propensity scores are used to create a distribution of the features for patients of the other medical providers to match a distribution of the features for the patients of the given medical provider. The propensity scoring component 116 applies the propensity score to the patient data of the other medical providers to weight the data of the patient records such that the weighted data for the patients of the other medical providers (excluding the given medical provider) closely resembles the non-weighted data for the group of patients of the given medical provider.

The regression modeling component 118 provides an interface for the operator to estimate the effects of the service provider on observed outcomes. In the example of benchmarking for medical service providers, the regression modeling component 118 may estimate the relative likelihood that a patient of the given medical provider would experience an identified outcome (such as expected patient readmission rate within 30 days) as compared to if the patent had been treated by the other medical providers in the record database 110. The regression modeling component 118 receives weighted data weighted by the propensity scoring component 116.

The benchmark comparison component 120 provides an interface for determining an outlier probability for each service provider for each outcome to identify underperforming and overperforming service providers. The outlier probability determines whether this service provider should be expected to have an elevated (or reduced) outcome relative to other service providers, based on the mix of cases for that particular service provider.

The dynamic updating component 122 provides an interface for updating the propensity scoring, regression modeling, and benchmark comparison, as the records in the service provider record database 110 are updated, including as new records are introduced into the record database 110 and as existing records in the record database 110 have changed.

The data output component 124 provides an interface to allow the operator to select the format and style for presenting analysis machine results. In some embodiments the data output component 124 includes a tool for selecting and formatting data produced by the analysis machine. In other embodiments the tools in the data output component 124 allow the operator to select and manipulate various visualization tools such as charts and graphs to assist interpretation and understanding of analysis machine results.

The benchmarking system and methods according to embodiments of the present invention described herein may be implemented utilizing any suitable hardware, firmware (e.g. an application-specific integrated circuit), software, or a combination of software, firmware, and hardware. The various components of the benchmarking systems and methods may be incorporated in various servers, controllers, engines, and/or modules (collectively referred to as servers), which may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of the benchmarking system and methods may be integrated into a single computing device, or distributed across one or more other computing devices without departing from the spirit and scope of the exemplary embodiments of the present invention. The server may be web-based.

The benchmarking system and methods may be accessed on a computing device through a portal to a web-based, Internet-based, or online server.

The benchmarking systems and methods may be implemented in a computing device that may be any workstation, desktop computer, laptop or notebook computer, server machine, handheld computer, mobile telephone or other portable telecommunication device, media playing device, gaming system, mobile computing device, or any other type and/or form of computing, telecommunications or media device that has sufficient processor power and memory capacity to perform the operations described herein. In some embodiments, the computing device may have different processors, operating systems, and input devices consistent with the device.

The computing device may be one of a plurality of machines connected by a network, or it may include a plurality of machines so connected. The network may include a network environment having one or more local machines, clients, endpoints, or nodes in communication with one or more remote machines or servers via one or more networks. The network may be a local-area network (LAN), e.g., a private network such as a company Intranet, a metropolitan area network (MAN), or a wide area network (WAN), such as the Internet, or another public network, or a combination thereof.

The computing device may include a network interface to interface to the network through a variety of connections including, but not limited to, standard telephone lines, LAN, WAN, broadband connections, wireless connections, or a combination of any or all of the above. Connections may be established using a variety of communication protocols. In one embodiment, the computing device communicates with other computing devices or servers via any type and/or form of gateway or tunneling protocol such as Secure Socket Layer (SSL) or Transport Layer Security (TLS).

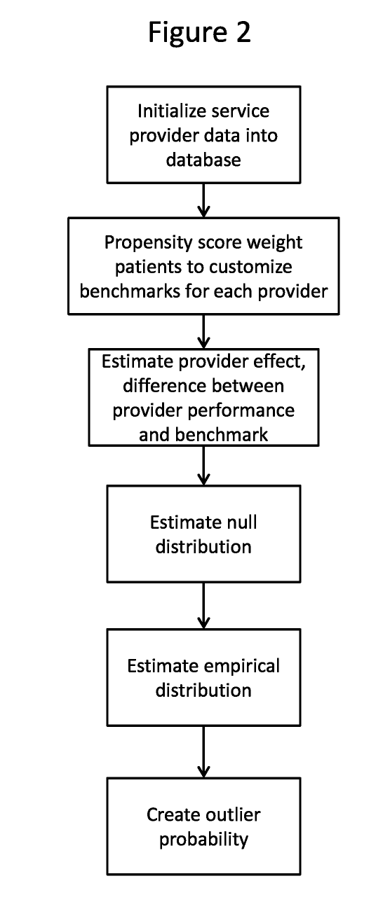

FIG. 2 depicts a flow diagram of steps performed using a service provider benchmark system according to one embodiment. The steps include an initialization of a service provider database identifying the effects of the service provider on various outcomes based on a regression model, and a benchmark comparison for each outcome. The benchmark comparison steps include estimating a null distribution assuming no outliers in the data, an empirical distribution estimate, and an outlier probability.

In one embodiment in the example of benchmarking for medical services, benchmarking hospitals involves the steps of assembling for each hospital a collection of patients treated at other hospitals who resemble the hospital's patients, contrasting the outcomes for each hospital's patients with the outcomes for their benchmark patients, and calculating the false discovery rate for each hospital, the probability that the hospital exceeds their benchmark.

### Initialization for Each Provider:

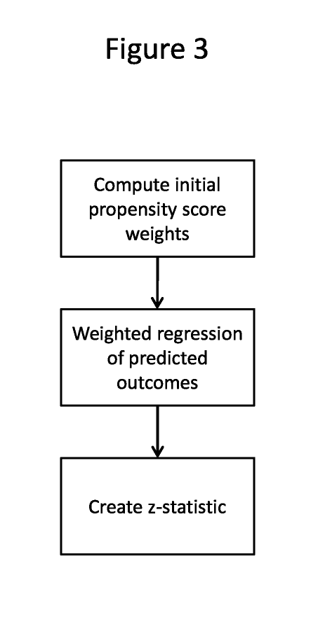

FIG. 3 depicts steps of an initialization process of the service provider benchmark system according to FIG. 2. As an initial setup, for each service provider among a plurality of service providers, the following elements are created: (1) propensity score weights for each patient treated by other service providers, (2) a regression model providing an estimate of the effects of the service provider on outcomes, and (3) a z-statistic that measures the effect of the service provider on the identified effect.

To construct a benchmark set of patients for a hospital, weights are assigned to patients treated at other hospitals so that, after weighting, those patients have features that collectively resemble the hospital for which the benchmark is being constructed. In the example of benchmarking for medical services, retrospective observational medical record data are selected by an operator for a first medical provider from a plurality of medical provider records. First, for each medical provider, excluding the first medical provider under test, their patients are assigned propensity scores to reweight their patient data. The propensity scores are used to create a distribution of the features for patients of the other medical providers to match a distribution of the features for the patients of the first medical provider. An example of a dataset for various medical providers is listed in the table below. The features at issue for the propensity scoring can include data on categorical features (such as percentage of male or female patients, age distributions, race, occupation, city of residence, prior diagnoses, prior prescriptions, reasons for admission, and the like), numerical measurements (such as temperature, blood pressure, and cholesterol levels, and the like), and patient outcomes (such as hospital readmission within 30 days). The propensity score provides a likelihood of a patient being treated by the first medical provider being benchmarked.

Propensity scores are created for each provider's patients in the plurality of medical providers, thereby enabling benchmarking data sets for each provider. By way of an example, the set of propensity scores when benchmarking Provider 1001 in the table below is created to reweight patients seen by all other providers, for example Provider 2003, and Provider 3007. Analogous sets of propensity scores for benchmarking_Providers 2003 and 3007 are created in the same way.

| | | | X₂ | |
|---|---|---|---|---|
| | | X₁ | (An example | |
| | | (An example | numeric | Y |
| | | categorical | measurement such | (An outcome |
| | | feature such | as temperature, | such as hospital |
| Subject | Provider | as race, occupation, | blood pressure, | readmission |
| ID | ID | city of residence) | LDL) | within 30 days) |
| 1 | 1001 | A | 101.1 | 1 |
| 2 | 1001 | A | 97.3 | 0 |
| 3 | 2003 | A | 109.9 | 0 |
| 4 | 2003 | B | 103.1 | 1 |
| 5 | 3007 | A | 103.3 | 0 |
| . . . | | | | |

The propensity scores may be created by a propensity scoring software component that determines and assigns a propensity score to each patient in a patient record database containing patients treated by multiple medical providers.

In one embodiment, the propensity scores may be created as follows. Patient records associated with multiple medical providers are populated in a database or table, for example, by the propensity scoring software component. Initial “seeding” propensity scores for each provider's patients and residuals are assigned. In one embodiment, initial seeding scores are calculated by dividing the number of patents for a given provider (e.g., Provider 1001) by the total number of patient records across all providers. The residual for each patient record is calculated by subtracting the initial seeding scores from a provider indicator identifying whether the patient is associated with the given provider or not. The provider indicator can be assigned a value of 1 if the patient is one of the given provider's patients, and assigned a value of 0 if the patient is not one of the given provider's patients. The provider indicator may be populated as a column in the database or table.

In addition, indicator functions are calculated and populated in the database, for example, as columns in the database. Indicator functions identify whether the patient record has a particular feature (e.g., male, age<16, age<17, high blood pressure, anti-depressant prescription, body mass index>30). The indicator function is assigned a value of 1 if the patient record met the criteria and a value of 0 otherwise.

After initial seeding propensity scores are determined, the largest absolute correlation between any indicator function column, or product of indicator function columns, and the residual column. For the following formulas, tᵢ represents the 0/1 indicator of patient i being treated by the given provider, pᵢ is the estimated probability that patient i is one of the given provider's patients, Iⱼ is the j^{th} indicator function, and n is the number of patients. To determine the extent to which any two columns are correlated, in order to identify the largest absolute correlation, the following formula is employed: rⱼ=Sumᵢ((tᵢ−pᵢ)(Iⱼᵢ−mean(Iⱼ))/((n−1)sd(t−p)sd(Iⱼ)).

After identifying the column j most correlated to the residual column, the propensity scores for all patients are adjusted based on the identified column. In adjusting the propensity scores the following formula is employed: pᵢ/(1−pᵢ)=pᵢ/(1−pᵢ)×exp(δ×sign(rⱼ)×Iⱼᵢ), where δ is a tuning parameter set to a small number such as 0.001. Using the new propensity score that results from the adjustment process, weights are applied to each of the other service provider's patient records. The weights are calculated from the propensity score using the following formula: wᵢ=pᵢ/(1−pᵢ).

The aggregate weighted data for patients of other service providers are compared against the aggregate data for patients of the given service provider to determine whether the two data sets are sufficiently similar (“optimally balanced”). If the data sets are not optimally balanced, the process is repeated using the updated propensity scores and residual as opposed to the initial seeding scores. The largest absolute correlation is determined in the same manner as before using the new residual, adjusting the patient propensity scores, determining new patient data weights based on the newer propensity scores, and comparing the weighted service provider data for other providers to the non-weighted service provider data to the given service provider determine whether the data sets are now optimally balanced. This process repeats until the data sets are sufficiently similar.

In a second step of the initialization, an estimate of the effect of a given service provider on patient outcomes is calculated. To accomplish this, a weighted regression model fit predicting patient outcomes (such as opiate prescription, infection, hospital readmission) is created based on the weighted data from the propensity scores. The weighted regression model is created from a Boolean (0/1) indicator that the patient was treated by the provider being benchmarked or not, and all of the other patient features.

To compute a doubly robust estimate of the effect of the provider, and simultaneously adjust for remaining confounding, the system estimates_a propensity score weighted generalized linear model. Depending on the type of outcome the regression model will be an ordinary least squares model (for continuous outcomes), a logistic regression model (for 0/1 outcomes), a Poisson regression model (for count outcomes), or other standard statistical models appropriate for the type of outcome. The estimates are derived from maximizing the equations L(b,β)=Σᵢ₌₁ⁿwᵢ(yᵢ−fᵢ)²L(b,β)=Σᵢ₌₁ⁿwᵢ(yᵢ−fᵢ)² (for ordinary least squares), L(b,β)=Σᵢ₌₁ⁿwᵢ(yᵢfᵢ−log(1+exp(fᵢ)))L(b,β)=Σᵢ₌₁ⁿwᵢ(yᵢfᵢ−log(1+exp(fᵢ))) (for logistic regression), or L(b,β)=Σᵢ₌₁ⁿwᵢ(yᵢfᵢ−exp(fᵢ))L(b,β)=Σᵢ₌₁ⁿwᵢ(yᵢfᵢ−exp(fᵢ)) (for Poisson regression), where yᵢyᵢ is the patient outcome being studied and fᵢ=b₀+b₁I(providerᵢ=X)+β′xᵢfᵢ=b₀+b₁I(providerᵢ=X)+β′xᵢ, and X is the label for the provider currently being benchmarked. The doubly robust provider effect estimate is b₁b₁. We compute the z-statistic as

$$\frac{{b}_{1}}{standard error ( {b}_{1} )} \frac{{b}_{1}}{standard error ( {b}_{1} )} .$$

In a third step of the initialization, a z-statistic is extracted from the regression model that measures the effect of a service provider on an identified effect. The z-statistic is a measure of how much evidence there is that a particular service provider deviates from their benchmark for an identified outcome. The z-statistic can be calculated for each service provider and for a plurality of identified effects.

After the above processor is run for each service provider, a z-statistic is available for every provider for every patient outcome being measured.

After calculation, the analysis machine outputs the doubly robust adjusted provider effect and the z-statistic to another location where it will be used to evaluate the performance of this provider in comparison to other providers.

### Benchmarking Comparison:

Next, a benchmark comparison is created for each outcome (e.g., infection rates, opiate prescriptions, etc). An analysis for such steps is described in Efron, Bradley (2010). Large-Scale Inference. Cambridge University Press. ISBN 978-0-521-19249-1.

In a first step of the benchmark comparison, a “null distribution” is estimated. That is, the distribution of z-values that one would expect if there were no outliers in the data. An estimate of the variance of distribution of z-values is created based on the curvature of a histogram of the benchmark near a z-value of 0.

In a second step of the benchmark comparison, an empirical distribution is estimated. That is, the distribution of observed z-values from the measured data that was actually observed.

In a third step of the benchmark comparison, an outlier probability is computed as P(outlier|z)=1−f₀(z)/f(z) where f₀(z) is the null distribution and f(z) is the empirical distribution.

### Reporting:

For each service provider, a report card is created listing its observed patient outcomes, its benchmark outcomes, and the outlier probability, as shown in the table below. The Provider X column is computed as the percentage or mean of the features of patients treated by Provider X. The Benchmark column is computed as the mean of the weighted regression model predictions of what would have happened to the Provider X patients had they been treated elsewhere,

$$\frac{1}{{n}_{X}} {\sum }_{i = 1}^{n} I ( {provider}_{i} = X ) \hat{y} ( {x}_{i} ) \frac{1}{{n}_{X}} {\sum }_{i = 1}^{n} I ( {provider}_{i} = X ) \hat{y} ( {x}_{i} )$$

where ŷ(xᵢ)=g(fᵢ)ŷ(xᵢ)=g(fᵢ), where g( ) is a function that transforms the fᵢ computed in the weighted regression model onto the outcome scale.

| | | | Outlier |
|---|---|---|---|
| | Provider X | Benchmark | probability |
| 30-day readmission | 15.8% | 11.7% | 0.00 |
| Oxygen expense (90-day) | $12.63 | $5.30 | 0.94 |
| Oxygen prescribed (per 100) | 9.7 | 9.7 | 0.00 |
| Oxycodone supply (30-day) | 5.7 | 5.0 | 0.00 |
| Oxycodone supply (90-day) | 12.3 | 11.4 | 0.00 |
| Opiate supply (30-day) | 10.1 | 12.1 | 0.61 |
| Opiate supply (90-day) | 23.5 | 29.0 | 0.51 |
| Any opiate prescribed | 49.2% | 57.0% | 0.63 |

For each outcome, a report card listing providers with high outlier probabilities can be created. An example of such a report card for the rate of opiate prescriptions is shown in the table below.

### Rate of Opiate Prescription per 100 Discharges

| Provider | Provider | Benchmark | Outlier |
|---|---|---|---|
| ID | rate | rate | probability |
| A | 62.1 | 51.8 | 0.99 |
| M | 36.6 | 31.8 | 0.99 |
| S | 63.6 | 36.1 | 0.99 |
| V | 61.4 | 46.7 | 0.99 |

### Dynamic Updating:

The previous steps describe a static, one-time process of fitting the propensity score model, outcome regression model, and reporting. As described above, the service provider record database may be continually updated to continually or intermittently updated with additional service provider data over time, for example, as existing hospitals in the database treat additional patients, or as the hospitals have updated records for existing patients, or as new hospitals are to be included in the database, or as new treatments are provided. When new records appear in the database and there is an existing benchmark for that service provider, the observed patient outcomes for that service provider may be updated to include the new data in a computationally efficient manner, and may be compared to the existing benchmark results. That is the outcome regression model, outlier probabilities, and reports may be updated in an efficient manner with new records so long as the original propensity score model is used and is not updated.

However, as new records appear for a service provider with an existing benchmark, the mix of cases, services, patients, or clients of the particular service provider may change. In the example of hospitals, additional patients may be added to the records of a service provider, which then changes the proportion of patients having a particular categorical feature, numerical measurements, and/or patient outcomes. For example, the added patient records may be predominantly under the age of 17, and thus the proportion of patients under the age of 17 is greater in the updated patient mix than at the time the benchmark was created. The result is that the benchmark may no longer represent the features of the records for the service provider, and a comparison between the results for the updated service provider records and the original benchmark becomes less accurate.

In some embodiments, the benchmark for the service providers may be updated to incorporate the additional records for each of the service providers in the database. When new records appear for each of the service providers, however, to update each benchmark simultaneously using the static process described above would require substantial computation, and would be too slow to keep up with the pace of incoming data. For example, a database may contain hundreds or thousands of service providers with incoming data continually being added for each of the service providers over time. The most computationally burdensome part of static benchmarking is computing the propensity score model. Described below is a dynamic model that can incorporate additional records into the benchmark after a static model has been created.

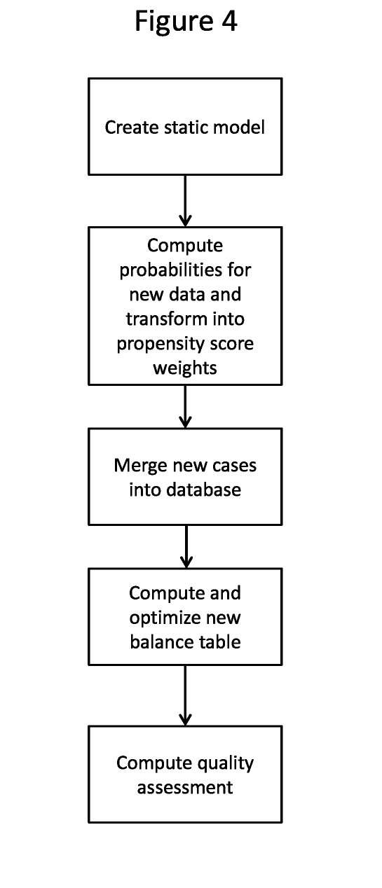

FIG. 4 depicts steps for dynamic updating of a service provider benchmark system according to one embodiment. To save on the computational expense of updating the benchmarks for each service provider, the dynamic model recomputes the propensity score model only when needed. To additionally save on computational processing power, the following process can operate continually in the background of a computer system or network. When there is available CPU capacity, the system will sort the providers by a “refresh” priority score measuring how important it is to check whether this provider's reports need updating. Service providers may be queued for updating their benchmarks, or for analyzing whether an update is necessary, based on their refresh priority score. Service providers with a higher refresh priority score will be placed higher in the queue to have their benchmark updated (or checked for updating) sooner than service providers with a lower refresh priority score. The refresh priority score can include a combination of the following elements for a given service provider:

(1) the number of new patient records that have entered the database for the service provider since the last time the propensity score model was created. A large number of new patients may warrant a higher priority;

(2) the percentage increase in the number of patient records that have entered the database for the service provider since the last time the propensity score model was created. Providers that have a large increase in the number of patients records may receive a higher priority, such as new providers that previously had few records;

(3) the number of new patient records, or the percentage increase of records, that have entered the database for the service provider having a particular feature of interest. For example, it may be determined that a particular feature has a relatively large impact on outcomes (such as being prescribed a certain medication), and may warrant a higher priority;

(4) whether the service provider was previously an outlier, and the severity to which they were an outlier. It may be desirable to refresh outlier providers for particular outcomes at a faster rate than service providers that are closer to the average for that outcome; and

(3) the quality of the existing propensity score model. Providers for which high quality benchmarks were difficult to construct may receive higher priority.

After sorting the providers by the priority score, for each provider, the following steps are performed to update the dynamic model.

In a first step, from the existing propensity score model, the probability that each new patient in the database would be treated by a given provider (e.g., Provider X) is computed. The probabilities are transformed into propensity score weights.

In a second step, the new cases are merged with the newly computed weights into the database containing the old cases.

In a third step, a new balance table is computed and is measured to determine if the table is optimally balanced. That is, if the new balance table is sufficiently similar to the data for the given provider, using the procedure described above.

In a fourth step, a measurement of the quality of the benchmark is computed. This quality measurement compares the distribution of weighted comparison patients with the distribution of a given provider's (e.g., Provider X's) patient features. For example, the largest Kolmogorov-Smirnov (“KS”) statistic may be used, which measures the largest difference across all values in the distributions. As an example for a distribution involving age, the KS distribution would be the largest difference across all values of age in the cumulative probability. In one example, if 65% of Provider X's patients were men and the weighted benchmark set of patients had 70% men, then the KS statistic would be 0.05. Similarly, if 40% of Provider X's patients were under age 21 and 42% of the weighted benchmark patients were under 21 AND this is the largest difference for any choice of age then the KS statistic would be 0.02.

In a fifth step, if the quality of the benchmark is within a specified or user-defined threshold or tolerance (i.e., the benchmark is sufficient), then the outcome regression model is recomputed, outlier probabilities are updated, and new reports are generated. That is, if the quality of the benchmark is sufficient, the original benchmark is used and the computationally expensive propensity score model is not updated. In some embodiments, the specified threshold may be 1%, such that the propensity score model is not updated if the percentage point difference between the aggregate updated data for a given service provider is within 1% of the aggregate weighted data for patients of the other service providers.

In a sixth step, if the quality of the benchmark deteriorates beyond a specified or user-defined threshold or tolerance, then the propensity score model is refit, and the outcome regression model is recomputed, outlier probabilities are updated, and new reports are generated. That is, if the quality of the benchmark becomes insufficient, the propensity score model is updated. In some embodiments, the specified threshold may be 1%, such that the propensity score model is updated if the percentage point difference between the aggregate updated data for a given service provider is outside of 1% of the aggregate weighted data for patients of the other service providers.

In some embodiments, different scheduling methods may be used to determine when to update the propensity score model. In some embodiments, each service provider may be updated in turn based on the amount of time since the last update for the service provider. The service provider which has waiting the longest time for an update may be updated first, and so forth. In some embodiments, each service provider may be updated when a given threshold of new records appears. In some embodiments, the updates may occur based on the size of the service provider.

Modifications of the above described embodiments are possible. The benchmark system and methods can be applied to many different industries and service providers. Such industries and service providers include medical (e.g., with service providers including physicians, nurses, pharmacists, clinics, hospitals and the like), emergency responders (e.g., with service providers including fire, police, emergency medical technicians, and the like), education (e.g. with service providers including teachers, principals, and other educators), and telecommunications (e.g., with service providers including wireless phone service providers, internet service providers, media service providers, cable, and the like), as well as many others.

### Application Example for Medical Treatment Providers:

The following describes an application of the benchmarking system and methods described above for benchmarking medical treatment providers, based on a study of real-world hospital data for 26 hospital complexes in 98 municipalities. In this application example, benchmarking was applied to compare patient outcomes of mortality and readmission rates across the various hospitals.

As part of the initialization for each provider, data on all patients admitted to the hospitals for circulatory system diagnoses (ICD10 chapter 9) between 2011 and 2015 was recorded and included in a service provider record database. There were 363,460 that met these criteria. There were 646 unique ICD10 codes observed in the data, but 50% of those diagnosis codes appeared in 30 or fewer patients across all hospitals over the five years of the study period. To focus the study on more prevalent diagnosis categories and to ease the process of finding patient cases that match across hospitals, the recorded data included only those ICD10 codes assigned to at least 400 patients. The application example excluded patients with ICD10 code I999 (unspecified circulatory disorder). The application example retained 91% of the patient admissions for the study, totaling 331,513 patients.

In the service provider record database, the application example recorded the primary admission diagnosis for each patient, an identifier for the hospital admitting the patient, and an identifier of the patient's municipality of residence. For each patient, the following information was recorded: age, sex, and comorbidity history including ischemic heart disease, diabetes, hypertension, chronic obstructive pulmonary disease, connective tissue disease, ulcers, liver disease, dementia, chronic kidney disease, heart failure, cancer, and alcohol abuse. The data also include recorded history of prescriptions for NSAIDs, statins, SSRIs, antipsychotic, glucocorticoids, antidiabetics, antibiotics, nitrates, ACE inhibitors, angiotensin II receptor blockers, beta blockers, calcium channel blockers, diuretics, anticoagulants, antiplatelets, and ulcer drugs. Also recorded were the patient outcomes for benchmark the hospitals, which were the 30-day mortality post-discharge and the 30-day readmission.

### Methods

As described above, propensity scores for each service provider were created by applying weight functions to the other service providers in the study. For a hypothetical “Hospital A,” weighting meant mathematically solving for a weight function w(x) such that f(x|hospital=A)=w(x)f(x|hospital≠A)f(x|hospital=A)=w(x)f(x|hospital≠A), where xx represents the patient features, f(x|hospital=A)f(x|hospital=A) is the distribution of patient features at Hospital A, and f(x|hospital≠A)f(x|hospital≠A) is the distribution of features for all other patients not treated at Hospital A. The value of w(x)w(x) depends on the patient's features and will be larger or smaller depending on whether patients with features xx are more frequent in Hospital A or not in Hospital A.

Solving for w(x)w(x) yields

$$w ( x ) = K \frac{f ( hospital = A \vert x )}{f ( hospital \neq A \vert x )} = K \frac{p ( x )}{1 - p ( x )} w ( x ) = K \frac{f ( hospital = A \vert x )}{f ( hospital \neq A \vert x )} = K \frac{p ( x )}{1 - p ( x )} ,$$

where p(x)p(x) is the propensity score, which is the probability that a patient with features x received treatment at Hospital A. A patient ii not treated at Hospital A will receive weight p(xᵢ)/(1−p(xᵢ))p(xᵢ)/(1−p(xᵢ)). If a patient has features not frequently seen in patients at Hospital A then the weight will be near 0. If a patient has features that are frequent at Hospital A then the patient's weight will be large, especially if the patient's features are uncommon at the other hospitals. K is a constant that will cancel out in calculations of any weighted statistics.

An estimate for the propensity score p(x)p(x) was calculated from the patient data in the service provider record database. Generalized boosted modeling was used to estimate the propensity score. This modeling strategy is similar to logistic regression except that, rather than using the individual xs as covariates, a linear combination of basis functions is used. The following equation was used for generalized boosted modeling,

$$log ( \frac{p ( x )}{1 - p ( x )} ) = {β}_{0} + {β}_{1} {h}_{1} ( x ) + {β}_{2} {h}_{2} ( x ) + … ++ {β}_{d} {h}_{d} ( x ) .$$

Specifically, the functions hⱼ(x)hⱼ(x) are all piecewise constant functions of x and their interactions involving up to three patient features. This allows for the estimate of the propensity score p(x)p(x) to be flexible including non-linear relationships, threshold and saturation effects, and higher-order interactions. As a result, matching patient features on their entire distribution (not just their averages) is possible, as well as a match on combinations of patient features.

Estimating the propensity score without constraints may result in an unidentifiable and numerically unstable model. Boosting approximates the use of the lasso penalty when estimating models with maximum likelihood. That is, coefficients in are estimated by finding the βⱼβⱼ that maximize \hat{β}=arg max_{β}Σᵢ₌₁ⁿAᵢβ′h(xᵢ)−log(1+exp(β′h(xᵢ)))−λΣⱼ₌₁^{d}|βⱼ|\hat{β}=arg max_{β}Σᵢ₌₁ⁿAᵢβ′h(xᵢ)−log(1+exp(β′h(xᵢ)))−λΣⱼ₌₁^{d}|βⱼ|, where A is a 0/1 indicator of whether patient i was at Hospital A. The lasso or L₁ penalty is equivalent to constraining the total size of the coefficients. If λ=0λ=0 then maximizing is equivalent to standard logistic regression but with the hs as covariates. When λλ is large then the penalty forces all of the βⱼβⱼs to be close to 0 and will actually set many of the βⱼβⱼs to be equal to 0. Boosting iteratively relaxes the size of λλ, determining at each step which of the hⱼ(x)hⱼ(x) will have a non-zero coefficient, and includes them in the model. Even though the set of basis functions maybe extremely large, most of them have coefficients equal to 0 and never need to be computed or stored. The boosting algorithm iterates until the features of patients at Hospital A most closely resemble the features of patients at other hospitals. This approach has been shown to outperform alternative methods for estimating propensity scores.

The resulting set of \hat{β}ⱼ\hat{β}ⱼs and hⱼ(x)hⱼ(x)s are used to compute propensity score weights for patients at the other hospitals, using the formula

wᵢ=1/(1+exp(−\hat{β}′h(xᵢ))).

A regression model for contrasting mortality or readmission rates between Hospital A's patients and patients at other hospitals is calculated as

$$log \frac{P ( {y}_{i} = 1 )}{1 - P ( {y}_{i} = 1 )} = {α}_{0} + {α}_{1} {A}_{i} ,$$

where yᵢyᵢ is the 0/1 indicator for death or readmission within 30 days for patient i. exp(α₁)exp(α₁) gives the unadjusted odds-ratio. Instead of fitting this equation using a standard logistic regression, αα is estimated using a weighted log-likelihood where Hospital A's patients have weight 1 and the other patients have weight wᵢwᵢ, then exp(α₁)exp(α₁) will give a propensity score adjusted odds-ratio, removing any confounding due to x.

Doubly robust estimation is performed by including covariates and using weighted maximum likelihood to estimate αα. Since the propensity score weights uncorrelate the confounders from A, their inclusion can improve the estimate of α₁α₁ by reducing bias by removing any remaining imbalance between Hospital A's patients and the other patients.

The z-statistic is extracted from the weighted regression model as a measure of the difference between Hospital A's outcomes and the benchmark outcomes.

For each of the 26 hospitals in turn, a new propensity score model is refit and the doubly robust estimation is performed. This customizes a benchmark for each individual hospital and produces a z-statistic comparing each hospital's outcomes to each of their customized benchmarks, for a total of 26 z-statistics.

Next, false discovery rates were calculated to determine the probability that a hospital flagged as an outlier is actually not an outlier. Traditional statistical decision-making regards p-values less than 0.05 as signaling a difference. Were this same criterion used for judging whether a hospital differs from its benchmark, even if no hospital actually differed from its benchmark, it would be expected that one hospital would be flagged as an outlier (26 hospitals×0.05=1.3). Numerous methods exist for computing the false discovery rate, but many require a large number of test statistics in order to compute non-parametric density estimates. With 26 hospitals under test, the following equation is used to convert a set of p-values arranged in descending order, p₍ₘ₎, p₍ₘ₋₁₎, . . . , p₍₁₎p₍ₘ₎, p₍ₘ₋₁₎, . . . , p₍₁₎, into q-values as

$${q}_{( i )} = {min}_{{p}_{( j )} \geq {p}_{( i )}} \frac{{mp}_{( j )}}{j} {\sum }_{k = 1}^{m} \frac{1}{k} ,$$

where m is the number of comparisons (m=26 in our example). Any q₍ᵢ₎q₍ᵢ₎ that exceed 1 are set to equal 1. Using this method, the false discovery rate will be less than or equal to q₍ᵢ₎q₍ᵢ₎.

### Results

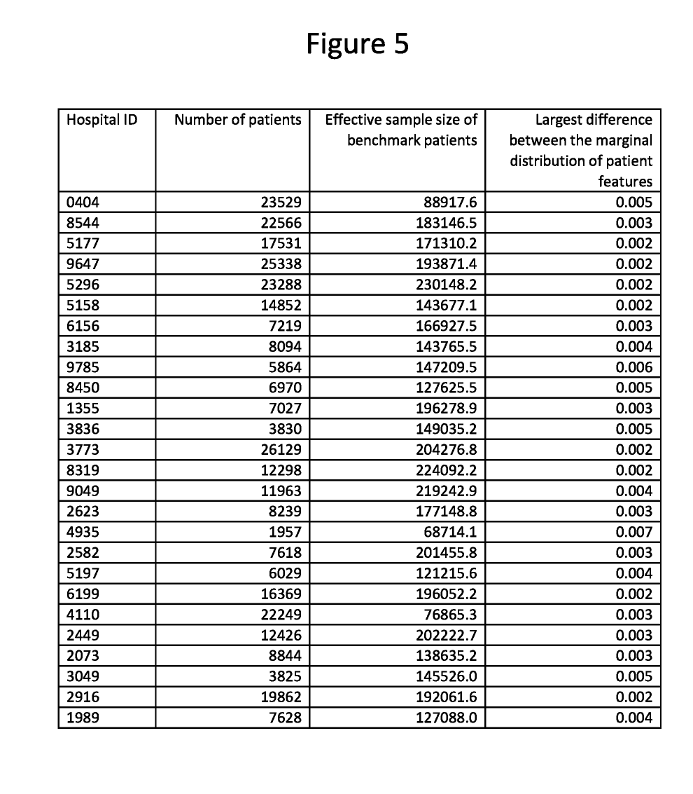

This section describes the results for benchmarking hospitals and municipalities using the methods described above. For each of the 26 hospitals, a high-quality benchmark set of patients was constructed consisting of tens of thousands of patients. FIG. 5 is a table showing the number of patients for each hospital, the effective sample size of the benchmark set of patients, and the largest difference in the patient features between the hospital and its benchmark. Each benchmark set of patients closely matched the associated hospital's patients, matching within 0.7 percentage points for all features for all hospitals.

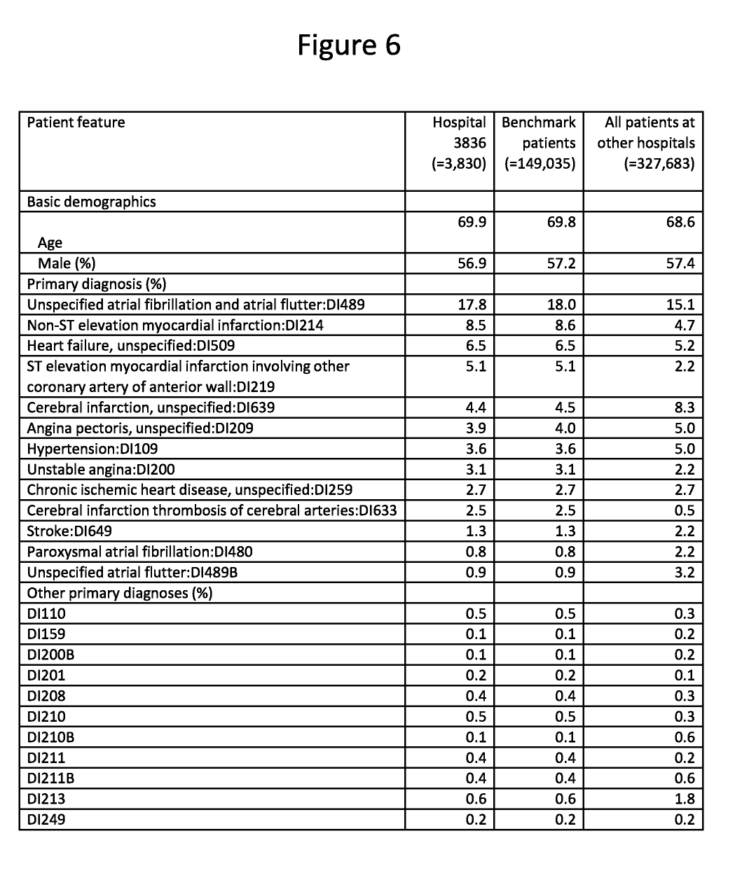

For hospital 3836 from FIG. 5, FIGS. 6-8 demonstrate the quality of the alignment of patient features between the Hospital 3836's patients and its benchmark set of patients. FIGS. 6-8 all show that the benchmark patients' features simultaneously align with Hospital 3836's patient features.

FIG. 6 provides a sample of patient features comparing Hospital 3836 to its benchmark. As shown in FIG. 6, the patient features include basic demographics, primary diagnosis, and other primary diagnoses. For all patient features, the hospital and benchmark features are in close alignment, and are frequently identical. Importantly, when compared to the collection of all other patients treated at other hospitals, the benchmark has customized the comparison set of patients so that non-ST elevation myocardial infarction cases are more prevalent in the benchmark set (8.6% of cases rather than 4.7%) and fewer paroxysmal atrial fibrillation cases (0.8% of cases rather than 2.2%).

Similar to FIG. 6, FIG. 7 also provides a sample of patient features comparing Hospital 3836 to its benchmark. FIG. 7 compares prior or concurrent diagnoses for the patients of Hospital 3836, known as comorbidities, to the benchmark for Hospital 3836. In addition to matching on primary discharge diagnosis, the benchmark patients also match Hospital 3836's patients on comorbidities as shown in FIG. 7.

Similar to FIGS. 6 and 7, FIG. 8 also provides a sample of patient features comparing Hospital 3836 to its benchmark. FIG. 8 depicts a comparison of prior prescriptions for a range of drug glasses for the patients of Hospital 3836 to the benchmark for Hospital 3836. FIG. 8 shows that the patients prescriptions for Hospital 3836 generally resemble the patient population for the 26 hospitals under test, but with slightly lower rate of antibiotic use (30% versus 36%) and slightly higher use of angiotensin II receptor blockers (15.1% versus 12.8%). However, the boosted propensity score model successfully constructed a benchmark set of patients that also matched Hospital 3836's patients on these features.

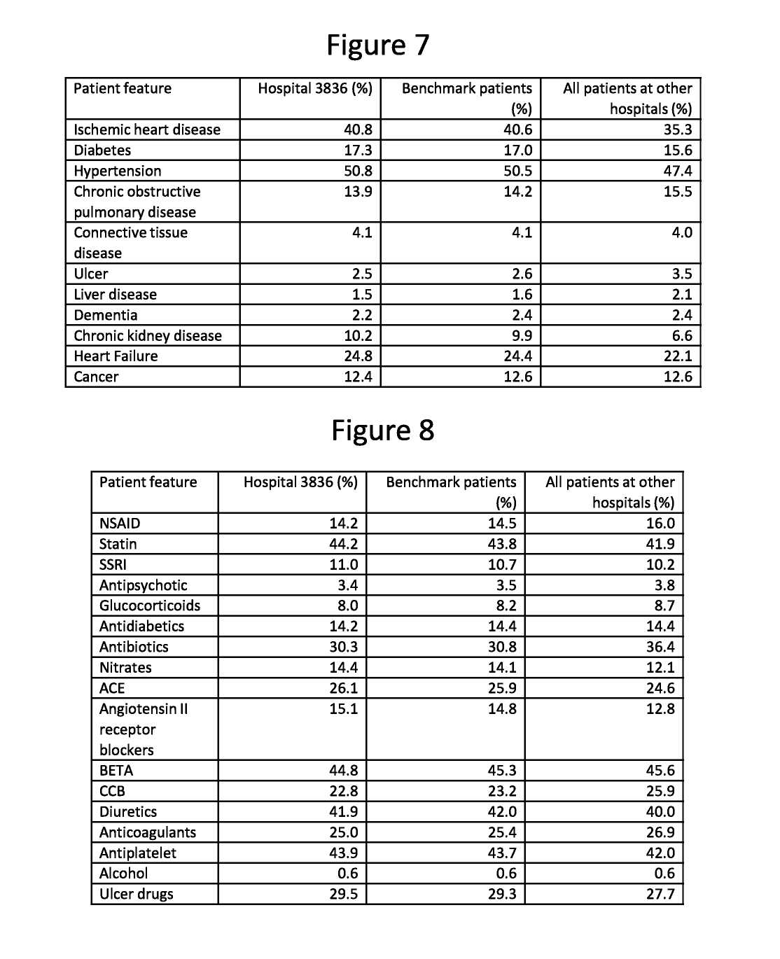

FIGS. 6-8 show that the marginal averages and marginal percentages match the constructed benchmark. FIG. 9 depicts a comparison of the age distribution of Hospital 3836's patients to the age distribution of the benchmark patients, showing near perfect alignment including the bulge at age 50 and 67. This demonstrates that the boosted propensity score model matches on the entire distribution including higher order interactions.

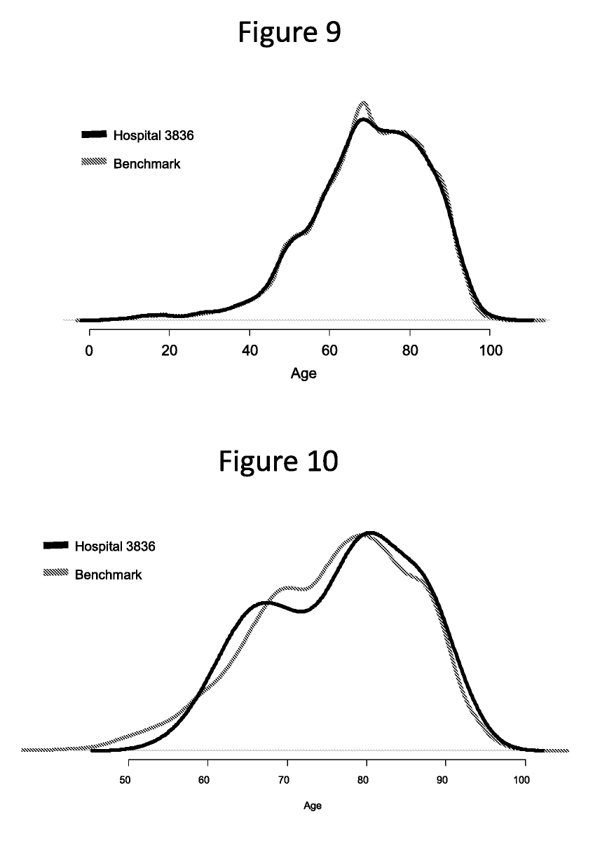

FIG. 10 depicts a comparison of a three-way interaction effect for Hospital 3836 as compared to its benchmark. In FIG. 10, the three-way interaction effect includes age, heart failure comorbidity, and statin use. FIG. 10 shows that the distributions align and demonstrate patient feature balancing for three-way interactions. The sample size of this group for Hospital 3836 is much smaller (n=148) so there is more noise in the density estimate, but the distributions still align closely.

Similarly to Hospital 3836, a customized benchmark was created for each individual hospital, similar balance tables for each of them was created, and it was confirmed that the quality of the benchmark was excellent in each case.

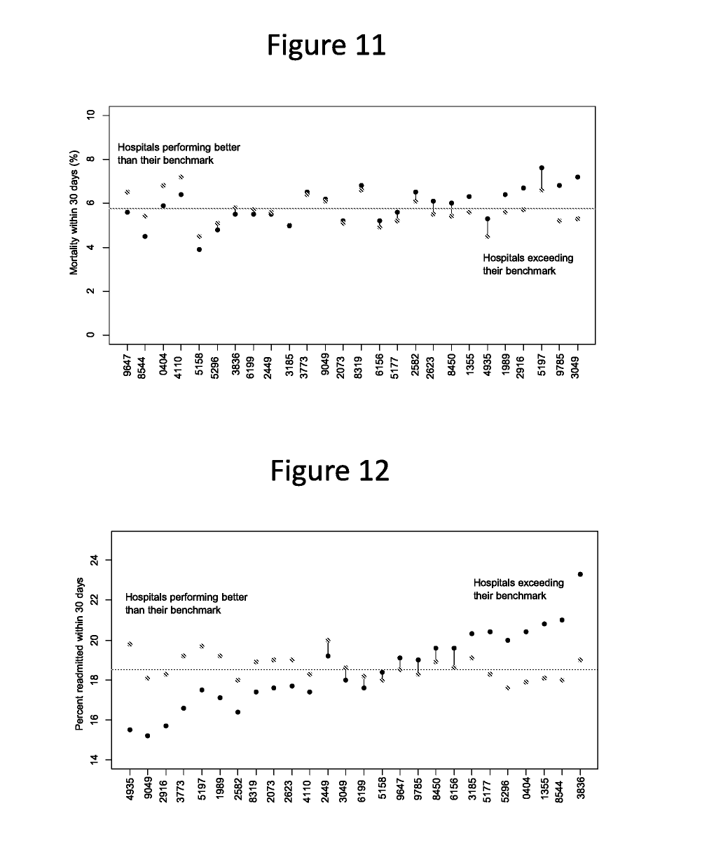

FIG. 11 depicts a comparison for each hospital's 30-day mortality rate to its customized benchmark mortality rate. Hospitals on the right of FIG. 11 have mortality rates that substantially exceed their benchmark. For example, Hospital 3049 had a 30-day mortality rate for circulatory system patients of 7.2%, while similar patients treated at other hospitals in the study had a mortality rate of 5.3%. The false discovery rate for this hospital was less than 1% indicating a high probability that this hospital is an outlier. At the other extreme, Hospital 9647 has a mortality rate of 5.6%, nearly a full percentage point lower than its benchmark of 6.5%. Also note that Hospital 8319, near the middle of FIG. 3, has a mortality rate that is relatively high, about one percentage point higher than the national average (shown by the horizontal line). However, its patient casemix is such that the benchmark calculates that this hospital should be expected to have an elevated mortality rate. Comparisons to the crude national average would normally highlight this hospital as an outlier. Hospitals with lines connecting the hospital and benchmark points mark those with false discovery rates greater than 5%, signaling that these may be statistically indistinguishable, though no public health standard has emerged on false discovery rate thresholds. Very large sample sizes in several comparisons produce small false discovery rates, even though the practical significance of the observed differences are slight. In FIG. 11, lines connect hospitals to their benchmark when the false discovery rate is less than 5%.

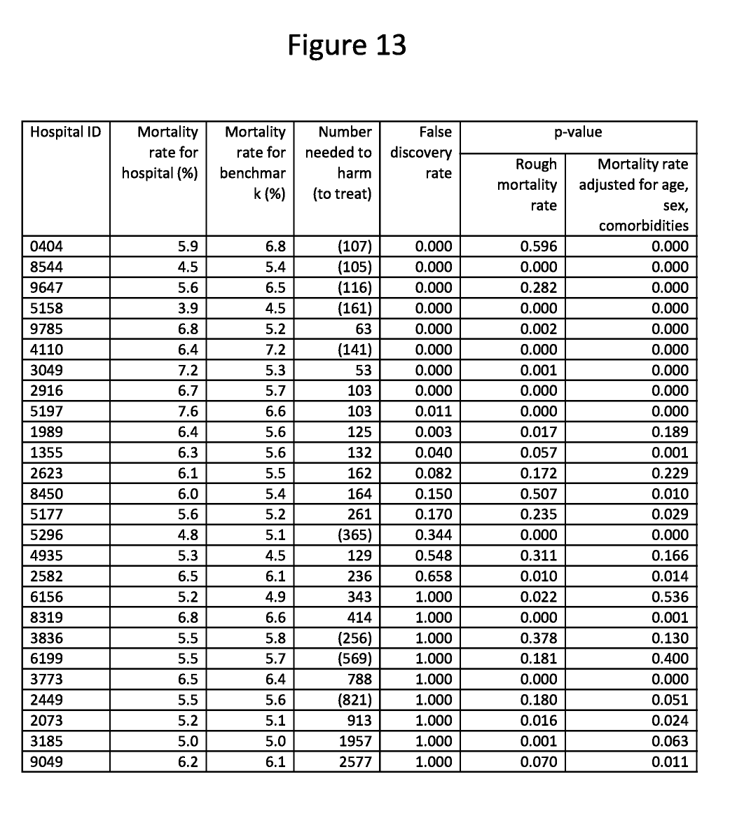

FIG. 12 depicts a comparison for each hospital's 30-day readmission rate to is customized benchmark mortality rate. Hospital 3836 (used to demonstrate the quality of the benchmark construction above), has a readmission rate exceeding 23%, far greater than the national average and its benchmark. Since the quality of the alignment between Hospital 3836's patients and its benchmark has been demonstrated, this difference in readmission rates cannot be due to any of the patient demographics, diagnoses, comorbidities, or prescriptions. Something else, such as other medical, organizational, economic, or social factors, must be causing this difference. Seven hospitals in total exceed their benchmarks with false discovery rates less than 5%. Hospital 4935, on the other hand, has a benchmark readmission rate near 20%, indicating that this hospital's case mix would be consistent with high admission rates. However, Hospital 4935 has among the lowest readmission rates of any hospital and more than 4 percentage points less than its benchmark.

One advantage of the benchmarking system described herein is that it is transparent in comparing a given hospital's patients with a closely aligned set of benchmark patients, for example, from other hospitals.

Next, a comparison between the benchmark system described herein for the hospital data was performed against the results of traditional analyses that may be used to attempt to identify outliers. Two traditional methods were used in the comparison. In the first traditional method, a rough comparison of hospital mortality and readmission rates with a national average. As described above, this type of comparison does not provide insight into whether deviations from the national average are due to a systemic difference within the hospital, or are due to a different patient mix for the hospital that has more or fewer outlier cases than average, or a combination of the two. In a second traditional method, an analysis was performed that adjusts for age, sex, and comorbidities, the latter either through a Charleson score or indicators of specific comorbidities. An unadjusted model was fit as

$$\frac{P ( Y = 1 \vert hospital = j )}{1 - P ( Y = 1 \vert hospital = j )} = {α}_{j} \frac{P ( Y = 1 \vert hospital = j )}{1 - P ( Y = 1 \vert hospital = j )} = {α}_{j} ,$$

where αⱼαⱼ is a hospital fixed effect, and a covariate adjustment model was fit as

$$\frac{P ( Y = 1 \vert hospital = j , x )}{1 - P ( Y = 1 \vert hospital = j , x )} = {α}_{j} + {β}^{′} x ,$$

where x is the collection of patient demographics, discharge diagnoses, comorbidities, and prior prescriptions. To flag outliers, the results of the unadjusted model and covariate adjustment model was converted from the log odds scale to the rate scale. For the covariate adjustment model, the equation was used to predict what would happen to the entire patient population if they had been treated at hospital j. That is, the expected rate for hospital j was computed by averaging over the empirical distribution of the patient features as

$$\hat{E} ( Y = 1 \vert hospital = j ) = \frac{1}{n} {\sum }_{i = 1}^{n} \frac{1}{1 + exp ( - ( {α}_{j} + {β}^{′} {x}_{i} ) )} .$$

For each hospital, it was tested whether Ê(Y=1|hospital=j)Ê(Y=1|hospital=j) differs from the population rate.

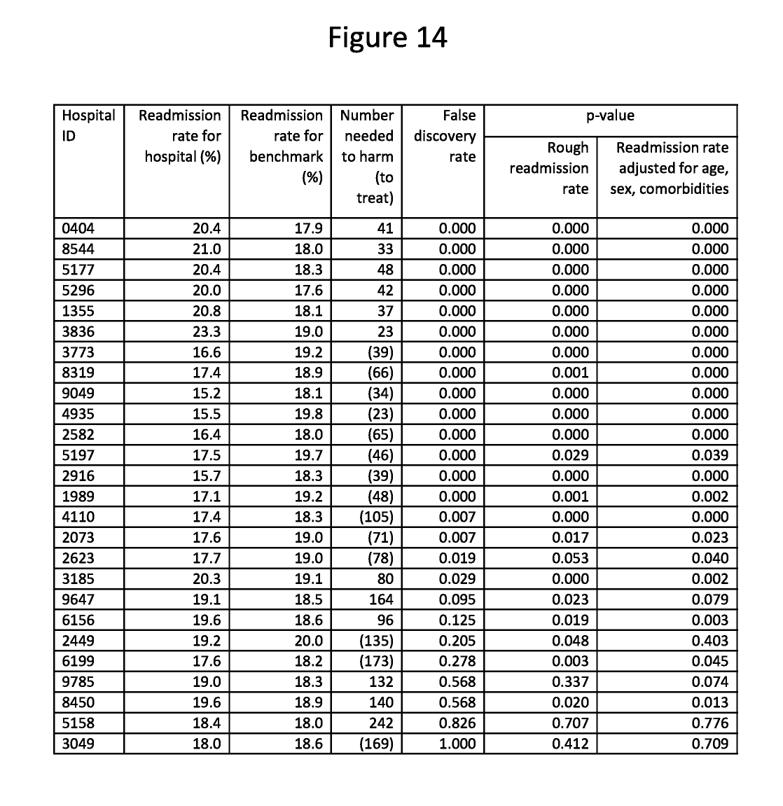

FIG. 13 depicts, for each hospital under test, a comparison of the hospital's mortality rate and the doubly robust estimate of their benchmark rate. The difference in those rates was converted into a number needed to harm (if the hospital had a higher mortality rate) or a number needed to treat (if the hospital had a lower mortality rate), computed as the inverse of the difference in the mortality rates. By this measure, Hospital 3049 stands out because for about every 50 patients, one more patient dies within 30 days than would have been expected had those 50 patients been treated at other hospitals. FIG. 13 also shows the false discovery rate and the p-values from the unadjusted model and the covariate adjusted model described above.

In particular, traditional approaches flag several hospitals as outliers for which the benchmark system identifies as not being outliers. The benchmark system calculates a false discovery rate near 1 for these hospitals, meaning that there is a high probability or near certainty that these hospitals are not outliers. Hospital 2073 is one hospital that demonstrates this, with its mortality rate nearly identical to the mortality rate of similar patients treated at other hospitals, yet traditional comparisons with unadjusted and covariate adjusted mortality rates flag this hospital as an outlier.

FIG. 14 depicts the analogous results of FIG. 13 for the 30-day readmission rates. For those hospitals with low false discovery rates, all methods agree that they are outliers. However, Hospitals 6156, 6199, and 8450 are identified via the unadjusted and covariate adjusted regression traditional model as outliers, yet the benchmark system herein estimates these hospitals as having high false discovery rates.

In accordance with the dynamic updating model described above, the data for the 26 hospitals in this study may be continually or intermittently updated with additional hospital data as new records are created. The service provider record database may be updated with additional data to provide the latest and most up to date information in the benchmarks. By updating the record database and benchmarks with new data, users can track trends over time and see the effects of new or revised treatment plans. For example, hospitals that were previously identified as outliers (e.g., as compared to their benchmarks) may be tracked to see if their performance changes over time. This may inform hospital administrators, other hospitals, and policy makers of whether new initiatives in high performing hospitals should be applied broadly to other hospitals, and whether remedial actions are warranted to improve the performance for underperforming hospitals, such as additional funding, revised policies, or changes in management.

As discussed above, as additional data is added to the service provider record database, the existing benchmarks may no longer sufficiently match the categorical features of the underlying hospitals. For example, referring to FIG. 9, additional patient records for hospital 3836 may be added having a different age distribution than is shown in the figure. By virtue of the different age distribution, the new data for hospital 3836 will decrease the quality of hospital 3836's benchmark. That is, the new data will introduce errors between the fit of the characteristics of the patients of hospital 3836 and the hospital's benchmark. Also as described above, the quality of the benchmark for hospital 3836 after additional records are added may be computed and compared to a user defined tolerance for determine whether to update the benchmark. In some embodiments, if the difference between the aggregate data for hospital 3836 and the aggregate data for the benchmark is larger than the tolerance, the quality of the benchmark s found to be insufficient and a revised benchmark is created.

In addition, the queuing methods described above may be used on the data to determine a priority for checking whether a given benchmark requires updating.

It shall be noted that features of the embodiments described above can be combined with features of other embodiments, mixed and matched to produce a variety of further embodiments.

While the present invention has been described in connection with certain example embodiments, it is to be understood that the invention is not limited to the disclosed embodiments, but is instead intended to cover various modifications and equivalent arrangements included within the spirit and scope of the appended claims, and equivalents thereof.

1. A system for dynamically evaluating service provider performance, the system comprising:
a processor; and
memory coupled to the processor, wherein the memory has stored thereon instructions that, when executed by the processor, causes the processor to:
establish an electronic communication channel with one or more electronic devices storing a plurality of record data;
receive, from the one or more electronic devices, first record data for a plurality of service providers and transmit the first record data to a database, wherein the first record data is transmitted over the electronic communication channel;
identify, from within the database, a portion of the first record data corresponding to a first service provider from among the first record data;
form a benchmark for the portion of the first record data for the first service provider by assigning weights to at least a portion of the first record data for service providers other than the first service provider to resemble the portion of the first record data for the first service provider;
receive, from the one or more electronic devices, second record data for the plurality of service providers and transmit the second record data to the database, wherein the second record data is transmitted over the electronic communication channel;
combine, within the database, the second record data with the first record data to form combined record data, wherein the second record data comprises data that was received after the benchmark was created;
compare the benchmark to the combined record data for the first service provider; and
sending data from the database to an electronic device based on the combined record data and benchmark.

2. The system of claim 1, further comprising determining whether the comparison of the benchmark to the combined record data for the first service provider is within a specified threshold.

3. The system of claim 2, wherein the comparison of the benchmark comprises comparing a distribution of features for the weighted data with a distribution of features for the first service provider.

4. The system of claim 3, wherein the threshold is set as 1% of the largest difference across all values in the distributions.

5. The system of claim 2, wherein when the comparison of the benchmark to the combined record data for the first service provider is within a specified threshold, compute a regression on the combined record data for the first service provider.

6. The system of claim 5, wherein the combined record data for the first service provide comprises an outcome for the first service provider, and
generating an outlier probability for the first service provider for the outcome based on the benchmark.

7. The system of claim 6 wherein the outlier probability is computed as as P(outlier|z)=1−f₀(z)/f(z) where f₀(z) is a null distribution and f(z) is an empirical distribution.

8. The system of claim 2, wherein when the comparison of the benchmark to the combined record data for the first service provider is outside of a specified threshold, forming a new benchmark for the first service provider by assigning weights to at least a portion of the combined record data for service providers other than the first service provider to resemble the combined record data for the first service provider.

9. The system of claim 2, wherein the service providers are hospitals.

10. A system for dynamically evaluating service provider performance, the system comprising:
a processor; and
memory coupled to the processor, wherein the memory has stored thereon instructions that, when executed by the processor, causes the processor to:
establish an electronic communication channel with one or more electronic devices storing a plurality of record data;
receive, from the one or more electronic devices, first record data for a plurality of service providers and transmit the first record data to a database, wherein the first record data is transmitted over the electronic communication channel;
form a benchmark for each of the plurality of service providers by assigning weights to at least a portion of the first record data for service providers other than a selected service provider to resemble the first record data for the selected service provider;
receive, from the one or more electronic devices, second record data for the plurality of service providers and transmit the second record data to the database, wherein the second record data is transmitted over the electronic communication channel;
combine, within the database, the second record data with the first record data to form combined record data, wherein the second record data comprises data that was received after the benchmark was created;
queue the benchmarks for the plurality of service providers to determine a priority for creating new benchmarks with the combined record data; and
sending data from the database to an electronic device based on the combined record data and benchmarks.

11. The system of claim 10, wherein updating the benchmarks with the combined record data comprises determining whether to update a benchmark for a given service provider.

12. The system of claim 10, wherein the priority for updating a benchmark for a given service provider is based on a percentage increase in the number of case records that have entered the database for the given service provider since the last time the benchmark was created for the given service provider.

13. The system of claim 10, wherein the priority for updating a benchmark for a given service provider is based on the number of case records that have entered the database for the given service provider since the last time the benchmark was created for the given service provider.

14. The system of claim 10,
wherein the combined record data comprises an outcome for each of the plurality of service providers,
for each of the service providers, generating an outlier probability for the outcome based on the benchmarks, and
wherein the priority for updating a benchmark for a given service provider is based on the outlier probability for the given service provider.

15. A system for evaluating hospital performance, the system comprising:
a processor; and
memory coupled to the processor, wherein the memory has stored thereon instructions that, when executed by the processor, causes the processor to:
establish an electronic communication channel with one or more electronic devices storing a plurality of record data;
receive, from the one or more electronic devices, record data for a plurality of hospitals and transmit the record data to a database, wherein the record data is transmitted over the electronic communication channel, and wherein the record data comprises a plurality of patients and an outcome for the patients of the plurality of hospitals;
identify, from within the database, a portion of the record data corresponding to a first hospital from among the record data;
form a benchmark for the portion of the record data for the first hospital by assigning weights to at least a portion of the record data for hospitals other than the first hospital to resemble the portion of the record data for the first hospital;
generate an outlier probability for the first hospital for the outcome based on the benchmark;
generate a report card listing an outcome for the first hospital, an outcome for the benchmark, and the outlier probability for the first hospital; and
transmit the report card to an electronic device for display.

16. The system of claim 15, wherein the listing of the outcome for the first hospital is an aggregate value for the patients of the first hospital, and wherein the listing of the outcome for the benchmark is an aggregate value for the patients of hospitals other than the first hospital

17. The system of claim 15, further comprising a plurality of outcomes, and wherein the report card lists the plurality of outcomes for the first hospital, the plurality of outcomes for the benchmark, and the outlier probability for each outcome for the first hospital.

18. The system of claim 16, further comprising
forming benchmarks for each of the plurality of hospitals,
generate an outlier probability for each of the plurality of hospitals for the outcome based on their respective benchmarks; and
wherein the report card lists the outcome for each of the plurality of hospitals, the outcome for each of the benchmarks, and the outlier probability for each of the plurality of hospitals.

19. The system of claim 16, wherein the report card organizes a listing of the providers by outlier probability.
